(19) United States
(12) Patent Application Publication
(10) Pub. No.: US 2014/0051945 A1
(43) Pub. Date: Feb. 20, 2014

(54) EXPANDED FREQUENCY ELECTROENCEPHALOGRAPHY/ELECTROCARDIOGRAPHY LIGHT APPARATUS AND METHOD OF USE

(71) Applicant: Terra Cotta Star LLC, (US)

(72) Inventors: Martha Marion Sarasua, Pensacola, FL (US); Michael Welch, Panama City, FL (US); Alice Merritt, Costilla, NM (US)

(73) Assignee: Terra Cotta Star LLC, Phoenix, AZ (US)

(21) Appl. No.: 13/795,728

(22) Filed: Mar. 12, 2013

Related U.S. Application Data
(60) Provisional application No. 61/609,714, filed on Mar. 12, 2012.
(60) Provisional application No. 61/666,498, filed on Jun. 29, 2012.

(51) Int. Cl. A61B 5/0402; A61B 5/0482; A61B 5/00; A61B 5/0476

(52) U.S. Cl. CPC A61B 5/0402; A61B 5/0476; A61B 5/0482; A61B 5/0006
USPC 600/301

(57) ABSTRACT

The invention is an apparatus and method that measures expanded frequencies of electromagnetic activity in a user's brain and heart. The apparatus includes a computer, a display screen, a software program for rapid measurement and digital display of the users electromagnetic brain and heart frequencies above about 500 Hz, a plurality of EEG sensors and an EKG sensor that are connected to the computer.

### CROSS REFERENCE TO RELATED APPLICATIONS

This application claims priority to and the benefit of the filing of U.S. Provisional Patent Application Ser. No. 61/609,714, entitled “Frequency Electroencephalography/Electrocardiography Light Apparatus and Method of Use”, filed on Mar. 12, 2012, and the specification thereof is incorporated herein by reference.

This application also claims priority to and the benefit of the filing of U.S. Provisional Patent Application Ser. No. 61/666,498, entitled, “Expanded Frequency Electroencephalography/Electrocardiography Light Neuro-Cardial Feedback Medical Treatment Apparatus and Method of Use” filed on Jun. 29, 2012, and the specification thereof is incorporated herein by reference.

### BACKGROUND OF THE INVENTION

1. Field of the Invention (Technical Field)

The present invention relates to an apparatus and method of use for expanding the frequency range for measuring time-based brain electromagnetic activity and heart electromagnetic activity.

2. Background

The heart and brain are the two major generators of time-dependent electromagnetic fields in the human body. The heart actually generates a larger temporal-spatial field than the brain. There is interaction between the heart electromagnetic field and the brain electromagnetic field that has yet to be explored deeply in conventional medicine. The temporal (time-dependent) properties of these two major electromagnetic field generators has only been measured in a limited sense through technology that was created approximately 90 years ago. The electrocardiogram (EKG) is the current device used to monitor the cardiac electrical signal through surface electrodes placed on the chest. The electroencephalogram (EEG) is the current device used to measure a limited range of brain electrical activity through surface electrodes placed on the scalp. The only major changes in this technology in 90 years has been the digitalization of the electrical signal allowing interface with computer display, storage, transmission and signal averaging techniques.

### Overview of Conventional EEG and EKG Technology

Electroencephalography (EEG) and electrocardiography (EKG) are long standing medical techniques for measuring time-based electromagnetic activity of the brain and heart, respectively, via surface electrodes placed on the scalp and chest. Conventional EEG and EKG have been in use as a standard of medical care for over 90 years. Both devices monitor electrical potential (voltage) as a function of time.

Conventional EEG measures a narrow frequency range from 0-30 Hz. The electrical activity of the brain is complex and remains poorly understood. The primary cells responsible for the generation of electrical signals in the brain are neurons. The neurons conduct signals through a complex mechanism of membrane-based ion channels which, when activated, generate an action potential. The signal is passed from one neuron to the next by two methods. Gap junctions allow direct coupling of the action potential of one neuron to the next. Synapses use a chemical transmission mode whereby the electrical signal from a first neuron causes release of chemical neurotransmitters into a spatial gap (synapse) between neurons. Binding of the neurotransmitter chemicals to receptors on a second neuron then stimulates an action potential carried along the length of this second neuron. The duration of a single action potential is in the range of about 1 msec ( 1/1000 of a second). Neurons have a resting potential measuring on average about −50-100 mV (millivolts) (negative on the inside of the cell). Hence, the resting neuron is said to be polarized. During an action potential this signal reverses to about +90-100 mV. Hence, the action potential is called a “depolarization”. After passage of the action potential there is a refractory (resting period) in milliseconds during which no additional action potential can be generated. The action potential thus results in a brief local electrical signal at the site of the activated neuronal with a relatively high amplitude, but short duration. At the synapse, an electrical signal called a postsynaptic potential is generated by binding of the neurotransmitter chemical to its receptor. A threshold postsynaptic potential results in generation of an action potential in the postsynaptic neuron. Postsynaptic potentials are only a few my in amplitude and can last for over 100 msec. Thus, they are of lower amplitude and frequency than the action potential. In addition to the neurons, the brain contains another cell type, the glial cells, which have recently been shown to utilize electrical and chemical signaling and may be capable of generating a portion of the brain's electrical activity.

The overall electrical activity of the brain is a very complex phenomena as the brain contains billions of neurons and glial cells. There are long-range and short-range mechanisms of communication between these cells and complex networks of cell communication and activity which underlie the complexities of human brain function. These mechanisms remain poorly understood and continue to be an enduring frontier of medical research.

EEG techniques involve the measurement of low voltage signals which travel from the brain to the scalp where the electrodes are placed. The electrical signals recorded by the surface electrodes of the EEG are thought to arise mainly from spatial and temporal summation of the relatively slow, low potential postsynaptic potentials with little to no contribution from the neuronal action potentials. The electrical signals monitored on the scalp are of low amplitude (about 10-110 μvolts; micovolts=1/1 millionth of a volt) due to attenuation of the signal as it travels from cortical brain matter through spinal fluid, meninges, skull and scalp musculature. In spite of the complexity of brain electrical function, a rhythmic pattern is observed in the conventional EEG with characteristic patterns for states of sleep and wakefulness. The origin of this rhythmic pattern remains unknown, but from animal studies it is thought to arise from neuronal pacemakers in the brain area known as the thalamus.

The conventional EEG has limited clinical EEG measurements to the frequency range of about 0-30 HZ. Bandwidth selection has separated various frequency ranges termed beta (13-30 HZ), alpha (8-13 Hz), theta (4-8 Hz) and delta (under 4 Hz). FIG. 1A illustrates a sample wave pattern with frequency, wavelength and amplitude. FIG. 1B shows an example of a normal EEG tracing.

Note that in addition to overall frequency variations, there are amplitude variations with the different EEG frequency regions, along with normal rhythmic variations in both amplitude and frequency. The conventional EEG has a long track record of being useful in characterization of this normal rhythmic brain pattern in the frequency range of about 0-30 Hz. It has proven of great clinical utility in characterizing normal human sleep cycles and diagnosing abnormalities such as seizure activity (abnormal bursts of electrical activity), sleep disorders, disorders that result in enhanced lower frequency activity such as drug effects, dementias, brain injury, and as a tool in defining brain death. However, the methodology remains limited as it only monitors a narrow range of brain electrical activity. For example, the high frequency action potentials are not “caught” by the current EEG machines.

Conventional EKG measures the electric potential (voltage) output of the cardiac electrical conduction system whose main function is to insure in regular contraction of the heart muscle to pump blood throughout the body with the relaxation phase in between contractions serving to return blood to the heart. The cardiac electrical conduction system begins with the sinatrial (SA) node located in the right atrium of the heart near where the large vein (vena cava) returns blood to the heart. The SA node is a small collection of highly specialized cells capable of self-generating and sustaining a regular electrical output. The SA node is thus the primary generator of the heart's electrical stimulation and acts as the cardiac time-keeper, maintaining a regular rhythm to the contraction/relaxation of the cardiac muscle. The SA node is thus termed the cardiac pacemaker. The signal generated by the SA node spreads out over a series of other specialized electrical conduction cells in a specific conduction pathway throughout the heart which can be viewed as the cardiac “electrical wiring system.” The electrical signal generated by the SA node spreads across the right and left atria through this conduction system signaling the atria to contract and pump blood into the right and left ventricles. The electrical signal then spreads through specialized conduction cells in the atrioventricular (AV) junction which includes an AV node and AV bundle (bundle of His). From this connection point between atria and ventricles, the electrical signal spreads through the left and right bundle branches carrying the electrical stimulus to the left and right ventricles resulting in their contraction to pump blood from the heart. The AV node thus acts as a bridge for the electrical signal to travel from atria to ventricles. The basis of the normal EKG is the measurement of this initiation and spread of electrical signal from the SA node through the AV node to the ventricles. See FIGS. 2A and 28.

Referring to FIGS. 2A and 28, the initial small wave is called the p wave and represents the atrial conduction process. The qrs complex is the sharper wave pattern that follows the p wave and represents the spread of the electrical signal across the ventricles. The final components of the EKG signal are the T and U waves which represent the recovery period of the electrical conduction system. The entire cycle from one SA activation to the next averages one second in the average human, representing a heart rate of about 60 beats per minute (1 beat per second); i.e. a frequency of 1 Hz. The heart is a powerful electrical field generator with a typical spatial distribution shown in FIGS. 3 and 4.

Embodiments of the present invention comprise an expanded frequency electroencephalography/cardiography apparatus and method of use. The apparatus and method integrates new technology with conventional EEG and EKG devices. The apparatus and method expands the frequency range of detection of the conventional EEG (currently limited to 0-30 Hz) to 500-1000 Hz (with upgrades to even higher frequencies). The expanded frequencies are above 30 Hz, preferably above 500 Hz and most preferably between about 500 Hz and about 1000 Hz and higher.

### SUMMARY OF THE INVENTION

One embodiment of the present invention comprises an apparatus for measuring electromagnetic activity in a user's brain and heart. This apparatus comprises a computer, a display screen, a software program for rapid measurement and digital display of the user's electromagnetic brain and heart frequencies above about 500 Hz, a plurality of EEG sensors, an EKG sensor, and the plurality of EEG sensors and said EKG sensor connected to said computer. The apparatus further comprises an integrated carrying case for storing the EEG sensors and the EKG sensor. The apparatus also further comprises an all-in-one carrying case for the computer and sensors. The apparatus optionally comprises a headset having the plurality of EEG sensors disposed on the headset. The user's electromagnetic brain and heart frequencies are measured and displayed between about 500 and about 1000 Hz. The apparatus is preferably portable. The computer is optionally a laptop or a tablet. The plurality of EEG sensors and EKG sensor are optionally connected to the computer via a peripheral USB device or are alternatively connected to the computer wirelessly. The software program preferably continuously measures the user's electromagnetic brain and heart frequencies for biofeedback.

Another embodiment of the present invention comprises a method for measuring electromagnetic activity in a user's brain and heart. The method comprises placing a plurality of EEG sensors on a user's head, placing an EKG sensor on the user's chest, transmitting a plurality of EEG signals to a computer server, transmitting an EKG signal to the computer server, measuring the user's electromagnetic brain and heart frequencies above about 500 Hz, and displaying the user's electromagnetic brain and heart frequencies on a display screen. The measuring of the user's electromagnetic brain and heart frequencies above about 500 Hz is preferably performed in real-time. The method of this embodiment optionally further comprises integrating the heart electromagnetic signal with the brain electromagnetic signal, detecting an abnormal pattern in either the heart electromagnetic signal or the brain electromagnetic signal. The EEG signals and the EKG signal is transmitted to a computer or is transmitted over the Internet to the computer server. The method optionally comprises tracking the user's electromagnetic brain and heart frequencies above about 500 Hz. The user's electromagnetic brain and heart frequencies are preferably measured between about 500 and about 1000 Hz. This method optionally continuously measures the user's electromagnetic brain and heart frequencies above about 500 Hz for providing biofeedback.

Further scope of applicability of the present invention will be set forth in part in the detailed description to follow, taken in conjunction with the accompanying drawings, and in part will become apparent to those skilled in the art upon examination of the following, or may be learned by practice of the invention. The objects and advantages of the invention may be realized and attained by means of the instrumentalities and combinations particularly pointed out in the appended claims.

### BRIEF DESCRIPTION OF THE SEVERAL VIEWS OF THE DRAWINGS

The accompanying drawings, which are incorporated into and form a part of the specification, illustrate one or more embodiments of the present invention and, together with the description, serve to explain the principles of the invention. The drawings are only for the purpose of illustrating one or more preferred embodiments of the invention and are not to be construed as limiting the invention. In the drawings:

FIG. 1A is a graph illustrating a conventional EEG sample wave pattern with frequency, wavelength and amplitude;

FIG. 1B is graph illustrating a conventional EEG tracing;

FIG. 2A is a graph illustrating a conventional EKG sample wave pattern;

FIG. 2B is a graph illustrating a second conventional EKG sample wave pattern with a diagram of a heart;

FIG. 3 is a drawing of a typical spatial distribution of the electrical field generating capacity of the heart;

FIG. 4 is a drawing of a typical spatial distribution of the electrical field generating capacity of the heart;

FIGS. 5 and 6 the full range of scalp locations available to a full clinical EEG;

FIGS. 7A-7H illustrate an embodiment of the present invention comprising a computer and EEG and EKG sensors;

FIG. 8 illustrates a flow chart of the software for one embodiment of the present invention;

FIGS. 9A-9D illustrate an embodiment of the present invention comprising a prevention reset apparatus;

FIGS. 10A-10D illustrate another embodiment of the present invention comprising a recovery reset apparatus;

FIGS. 11A-11D illustrate an embodiment of the present n comprising a sound- and light-based transformer unit; and

FIGS. 12A-12D illustrate an embodiment of the present invention comprising a thought amplification apparatus.

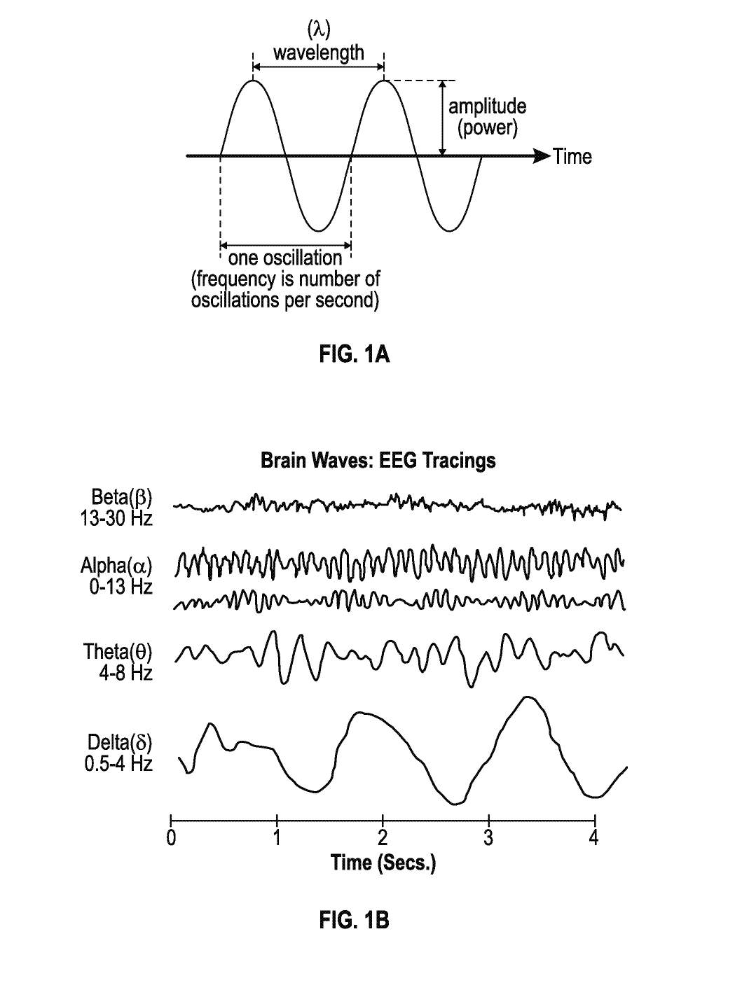

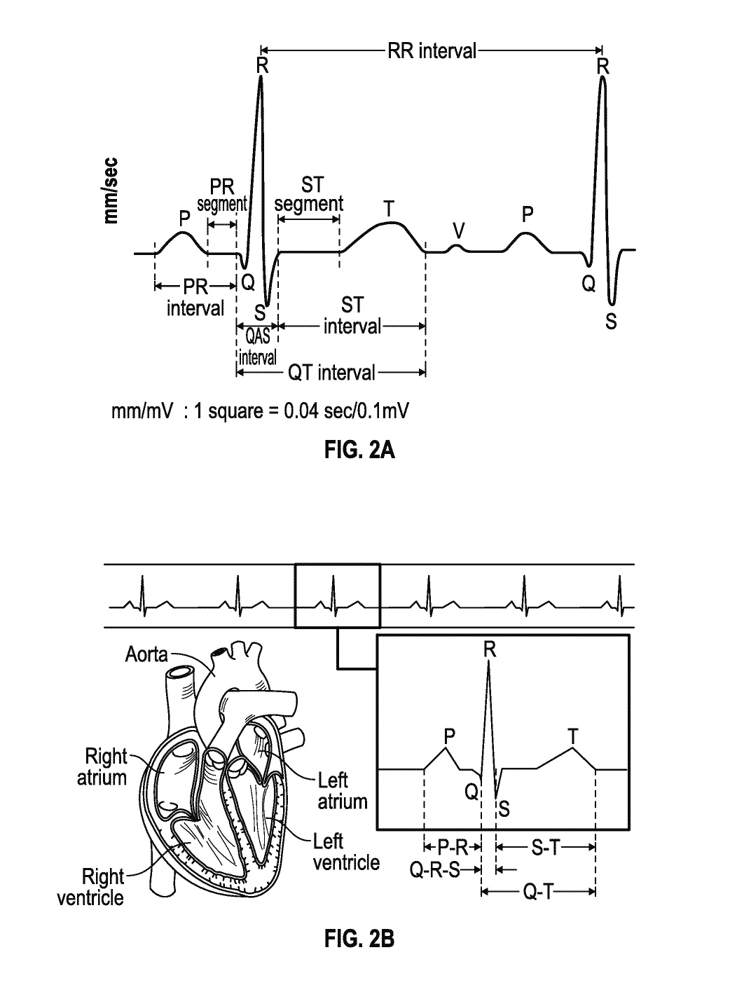

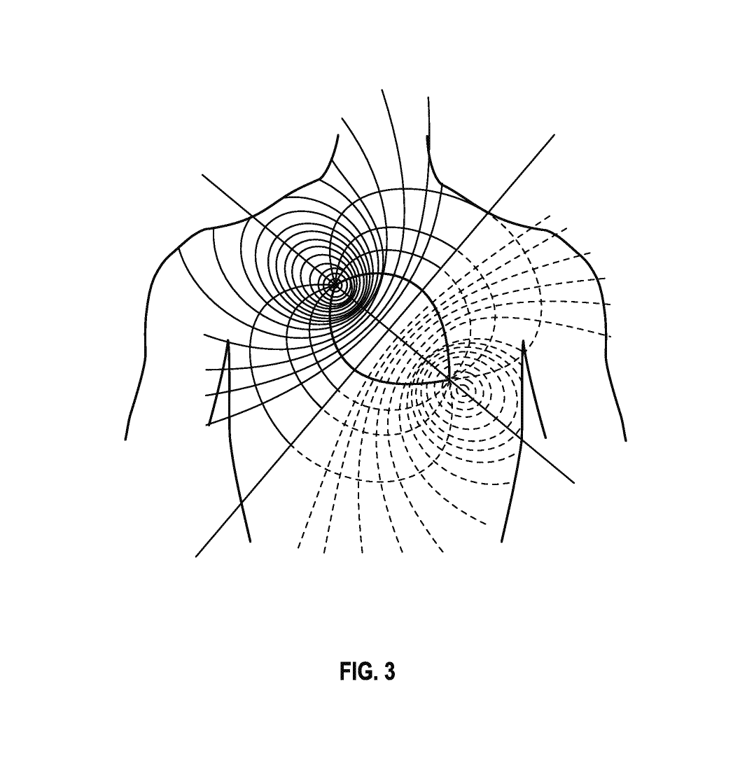

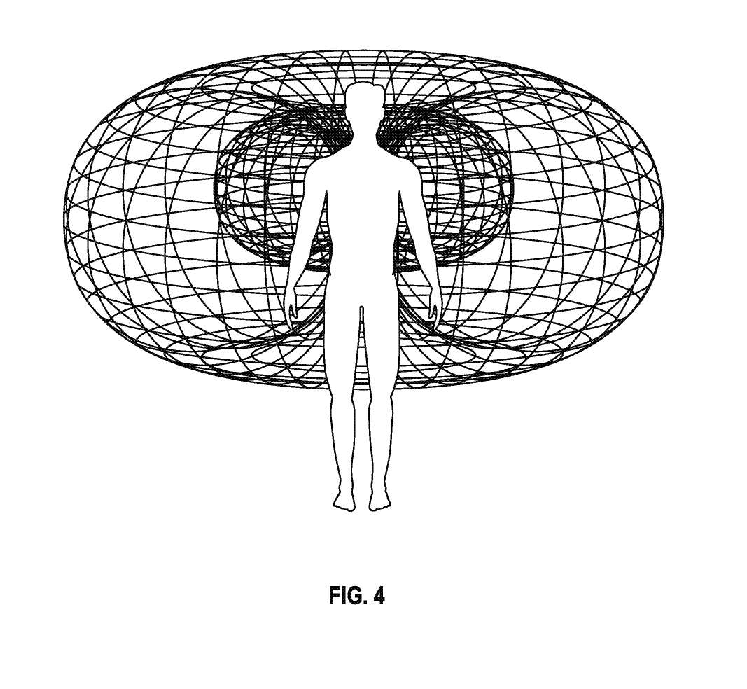

### DETAILED DESCRIPTION OF THE INVENTION

As defined in the specification and claims, “cardial-neural state” is a mathematical description (electrical field potential map) of an interaction between the electrical field of the heart and the electrical field of the brain at a given instant in time. Hence, it is the summation of the two electrical fields displayed in real-time.

As defined in the specification and claims, “cardial-neural feedback” is a biofeedback treatment that allows an individual patient to alter the cardial-neural state at will via computer feedback technology, i.e. adjust the interaction of the two fields through biofeedback.

An embodiment of the present invention comprises an expanded frequency electroencephalography/cardiography apparatus and method of use. The apparatus and method integrates new technology with conventional EEG and EKG devices. The apparatus and method expands the frequency range of detection of the conventional EEG (currently limited to about 0-30 Hz) to above 500 Hz (with upgrades to even higher frequencies). The expanded frequencies are above 30 Hz, preferably above 500 Hz and most preferably between about 500 Hz and about 1000 Hz. In addition, this embodiment of the present invention through software routines that involve specific mathematical algorithms, tracks temporal interactions between the cardiac and brain electrical fields. There are clinical advantages for the detection of higher frequency EEG patterns, such as for example, earlier detection of seizure disorders and traumatic brain injury, higher sensitivity in the detection of seizure disorders, open additional avenues for research that lead to other future clinical applications through correlating other brain and mental disorders with alterations in the high frequency range of brain wave activity. Coupling of the cardiac and brain field signals also allows access to new methods for clinical treatment of cardiac, neurological and psychiatric disorders. An embodiment of the present invention is a measurement device, and thus is used as a diagnostic and research tool. Earlier and more sensitive diagnosis of cardiac, neurological and psychiatric disorders leads to prompter treatment and improved clinical outcomes. The expanded frequency electroencephalography/cardiography apparatus of this embodiment expands the range of clinical measurement of brain wave activity into higher frequency brain regions which allows earlier and more sensitive detection of conditions such as seizures and traumatic brain energy. Earlier and more accurate detection allows for prompter treatment of these disorders thus improving clinical outcome.

Coupling of the cardiac and brain electrical fields also provides earlier and more sensitive detection of cardiac arrhythmias and electrical abnormalities produced by cardiovascular disease and cardiomyopathy, thus allowing access to treatment prior to the occurrence of major cardiac events. The heart and brain are the two primary electrical field generators in the human body. These fields are well-known to interact with one another. Thus, changes in the brain field affect the heart field and vice versa. This embodiment allows real-time detection and correlation of the interaction of these two electrical fields and opens avenues for research into the clinical significance of these interactions. In addition, the apparatus of this embodiment, as stated above, allows earlier and more sensitive detection of electrical abnormalities in the brain and heart resulting from a variety of clinical disorders such as seizures, traumatic brain injury, neuropsychiatric disorders including dementias, mood and anxiety disorders, cardiac arrhythmias, electrical abnormalities in the heart caused by coronary artery disease or cardiomyopathies. Earlier and more sensitive detection allows for earlier clinical intervention and improvement in clinical outcome in these disorders.

An embodiment of the present invention is also a treatment apparatus and method that extends the capability of current biofeedback and neurofeedback treatment as described below.

One embodiment of the present invention comprises an expanded frequency electroencephalography/electrocardiography apparatus and method of use that expands the measurement of time-based brain electromagnetic waves to frequencies above about 30 Hz, preferably above about 500 Hz and more preferably between about 500 Hz and about 1000 Hz. The apparatus is preferably light-weight, highly portable, and optionally wireless. The apparatus allows measurement of the conventional EEG frequency domains from 0 to 30 Hz, and expanded measurement capabilities from the upper limit of this range to about 1000 HZ and higher. Thus, the apparatus tracks an expanded range of brain electromagnetic function in order to provide earlier detection of seizures and earlier and more sensitive detection of brain injury and psychiatric disorders. The apparatus also provides measurement of the mathematical coupling between the spatial-temporal electromagnetic fields of heart and brain.

An embodiment of the present invention comprises a light-weight, less costly instrument that allows easier patient access and portability. The apparatus of this embodiment comprises a peripheral USB device that includes the EEG (electrocephalogram) and EKG (electrocardiogram) electrodes with connectors (optionally utilizing wireless technology), as well as transduction and digitization hardware. The apparatus further comprises a computer, preferably a laptop or tablet PC. The peripheral USB device optionally comprises a compartment holder for a practitioner's laptop or tablet PC. A signal, preferably a digitized signal is fed into a computer, preferably a laptop or tablet PC into which the apparatus software program has been downloaded. The software program allows rapid measurement and digital display of conventional clinical frequency domains (0-30 Hz), research zones (30-500 Hz) and higher frequencies (above 500 Hz). Upgrades are also available to continually expand the detection frequency range from about 1000 Hz and higher. The software of this embodiment comprises mathematical algorithms which measure the coupling of cardiac and brain electrical fields in time and space.

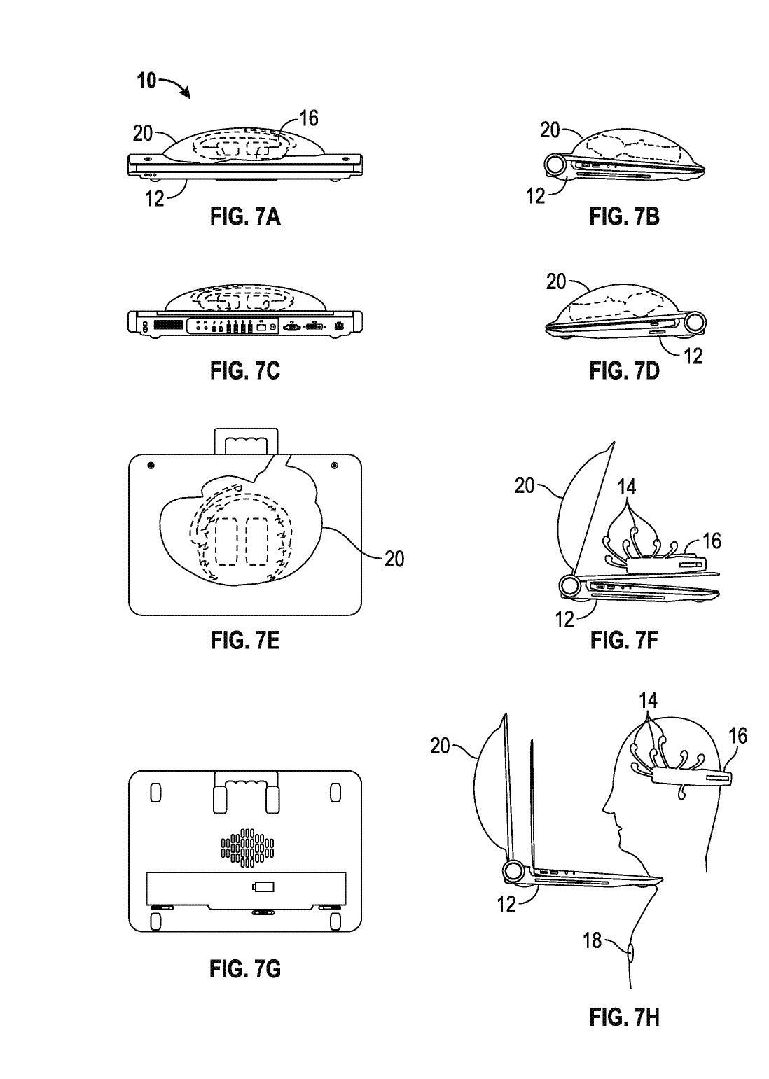

Referring to FIGS. 7A-7H, one embodiment of the present invention comprises a complete hardware/software solution for a convenient, transportable electroencephalograph and electrocardiograph. In one embodiment of the present invention, apparatus 10 comprises laptop computer and/or a docking pod 12 and EEG/EKG sensors 14 and 18, respectively. The apparatus preferably includes headset 16 comprising the EEG sensors 14. Headset 16 preferably comprises EEG sensors 14 and more preferably comprises about 5-20 saline sensors and most preferably about fourteen saline sensors that offer optimal positioning for accurate spatial resolution and gives the subject total range of motion. EKG sensor 18 is preferably small and lightweight. It is attached over the right atrium of the subjects chest and transmits EKG signals, preferably real-time, to computer 12 or over the Internet to a computer server that has been programmed to identify abnormal patterns. Headset 16 and EKG sensor 18 are optionally be stored with laptop computer 12 in an all-in-one carrying case, or are stored separately. Apparatus 10 is used to study brain waves within the typically frequency range, as well as frequencies far beyond what was previous thought to be of any significant clinical value. For example, this embodiment studies brain waves above about 30 Hz, preferably above about 500 Hz, and most preferably between about 500 Hz and about 1000 Hz. In one embodiment, apparatus 10 comprises carrying case 20 for storing the EEG/EKG sensors 14 and 18. Carrying case 20 is preferably an integrated carrying case and more preferably an integrated dome.

Integrated Software:

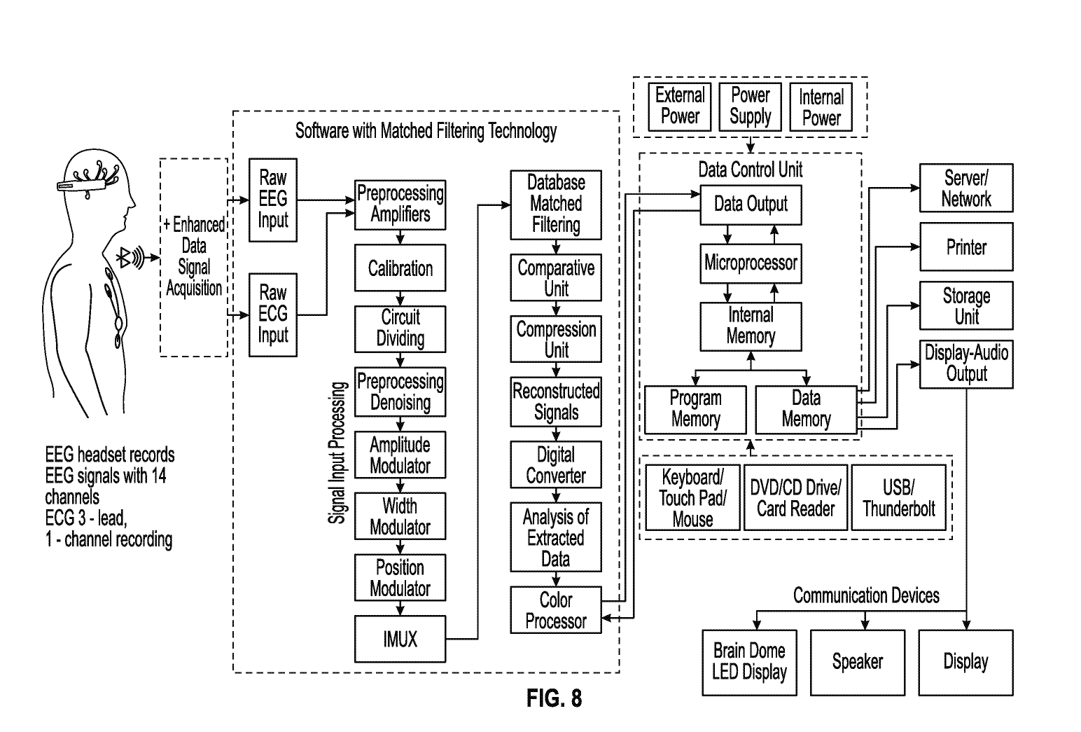

The software preferably runs independently from the computer. FIG. 8 illustrates a flow chart of the software. In one embodiment, the software is not copied to the computer's internal hard drive or any other attached hard drive. Sensors 14 and 18 of FIGS. 7A-7H preferably attach to the forehead, scalp, ear and chest of the subject. The sensors report and send raw waves to the computer either connected or wireless and preferably through wireless communication, but other communication apparatuses and methods are included. Matched filtering technology divides the raw waves signal by frequencies and by digital pulse amplitude modulation, while the software algorithms detect, correct, compensate for and tolerate all types of non-EEG noise. The resulting data is preferably displayed on a monitor of computer 12. These enhanced frequency signals allow access to new methods for clinical treatment of cardiac, neurological and psychiatric disorders.

Another embodiment of the present invention comprises an expanded neurofeedback stimulation and treatment apparatus and method. This embodiment comprises expanded software programming that allows performance of high frequency EEG neural feedback to effect stimulation of higher frequency brain regions across multiple brain regions. In addition, the expanded software programming integrates the cardiac electromagnetic signal with the electroencephalographic signal through a mathematical algorithm that links the base cardiac signal to increasingly higher frequency brain electromagnetic signals. The apparatus of this embodiment allows feedback in the frequency domains above about 30 Hz and preferably above 500 Hz with upgrades to even higher frequency realms.

This embodiment of the present invention uses the expanded frequency electroencephalography/electrocardiography apparatus described above for measurement and then incorporates software-based neural feedback aspects across a full frequency zone and coupled cardiac-brain feedback through the mathematical algorithms.

### Biofeedback

Biofeedback is a clinical technique in which learned consciousness mental control over physiological parameters such as heart rate (EKG), skin temperature, skin conductivity, respiration and EEG patterns is effected through measurement of these parameters coupled with mental feedback to alter the parameters through the conscious mind. The technique originated in the 1960's and has become an accepted clinical treatment tool for a variety of disorders such as hypertension, chronic pain, vascular headaches, tinnitus, cardiac arrhythmias, stress reduction, anxiety disorders, post-traumatic stress disorder and attention deficit hyperactivity disorder. The technique is also used to improve focus and concentration in normal humans with applications in industry and the military. EEG neural feedback (neurofeedback) is a specific form of biofeedback in which the individual learns to control brain wave (EEG) patterns consciously. The existing technology has been limited to altering the relative patterns of beta and alpha waves. The beta pattern is enhanced when an individual is consciously attending to an object or task. The alpha pattern is enhanced during relaxation techniques and meditation. Enhancing the ratio of beta/alpha has been useful in improving focus and concentration and treating attention deficit hyperactivity disorder (ADHD). The reverse enhancement of alpha/beta ratio results in relaxation and is helpful for stress management, reducing blood pressure, heart rate, respiratory rate and alleviating anxiety.

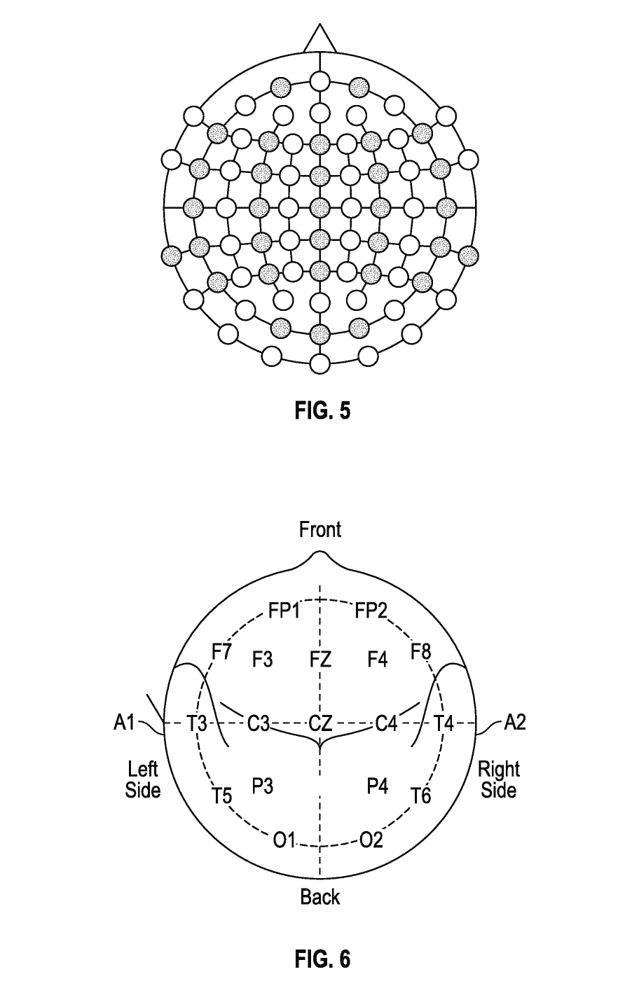

The conventional EEG neural feedback instruments allow feedback mechanisms only in this narrow frequency ranges assigned to beta (13-30 Hz) and alpha (8-13 Hz). In addition, conventional devices only utilize a few EEG electrodes over a limited scalp area, rather than utilizing the full range of scalp locations available to a full clinical EEG. See FIGS. 5 and 6 illustrating a full range of scalp locations available to a full clinical EEG.

Embodiments of the present invention improve neural feedback and biofeedback.

An apparatus and method of one embodiment of the present invention is capable of medical treatment based on new technology for neural-cardiac feedback. This embodiment provides a greater range of neural feedback options (higher frequency, expanded brain regions) along with the coupled cardiac-neural feedback and thus expands treatment capabilities of existing technology and allow improved treatment for traumatic brain injury, psychiatric illness and cardiac disease. The apparatus of this embodiment also provides improved treatment for the disorders listed above for which conventional neural feedback has already proven successful along with expanding treatment options to traumatic brain injury, all psychiatric illnesses and cardiac disease. Current biofeedback and EEG neurofeedback technology is limited to treatment of autonomic dysregulation such as anxiety disorders and hypertension by via manipulation of skin temperature or conductance (through basic relaxation techniques) or treatment of attention deficit hyperactivity disorder by changing the beta to alpha frequency patterns via EEG neurofeedback. The expansion of EEG neurofeedback into higher frequency zones allows access to balancing and enhancing these zones. The higher frequency EEG ranges are known to be affected in seizure disorders, traumatic brain injury and various neuropsychiatric disorders such as mood disorders, anxiety disorders and dementias. These effects manifest sooner in the high frequency zones, than in the conventionally measured EEG range. Many of these disorders cannot be treated by conventional biofeedback and EEG neurofeedback techniques. The apparatus of one embodiment of the present invention allows feedback techniques to be employed in these higher frequencies thus treating disorders not yet treatable via the conventional biofeedback and neurofeedback methods (e.g., seizure disorders, traumatic brain injury and various neuropsychiatric disorders such as mood disorders, anxiety disorders and dementias) to be treated. In addition, the feedback technology of embodiments of the present invention allows normal individuals to improve brain function by learning through feedback to enhance higher frequency brain activity. The feedback technology involves the user interacting with a figure or picture displayed on the computer that allows them to control his/her frequency patterns. For example, if EEG neurofeedback in higher frequency zones indicates that the user is having an anxiety attack, the user can then employ a relaxation technique to cairn down. The EEG neurofeedback then shows the response from the relaxation technique.

In another embodiment of the present invention, the apparatus couples the feedback of both brain wave patterns and cardiac electric fields for improved autonomic regulation via feedback techniques since the autonomic nervous system depends for its normal functioning on the proper coupling of brain and heart electric fields. The biofeedback techniques involve a personal interaction with a figure or picture displayed on a computer screen. Through the addition of new feedback techniques that allow an individual to adjust the field interaction between heart and brain at will, treatment options are expanded to include cardiac disorders and brain disorders related to autonomic nervous system dysregulation such as cardiac arrhythmias, parasympathetic dystrophy, gastrointestinal autonomic dysfunction, post-traumatic stress disorder, mood and anxiety disorders and neuroendocrine disorders such as adrenal insufficiency.

### Mathematical Algorithms:

The software programming of one embodiment of the present invention integrates the cardiac electromagnetic signal with the electroencephalographic signal through a software-based mathematical algorithm that links the base cardiac signal to increasingly higher frequency brain electromagnetic signals, allowing conventional feedback in the 0-30 Hz domain (but expanded over multiple brain regions) along with high frequency feedback in the frequency domains above about 500 Hz and preferably from about 500 Hz to about 1000 Hz with upgrade to even higher frequency realms available. Enhanced feedback through the cardiac-brain coupling is also achieved in order provide an innovative medical treatment apparatus and method for brain and cardiac disorders.

Referring to FIG. 8, the software preferably acquires a signal from the EEG headset and ECG sensor. The software then processes the input signal, which includes but is not limited to, calibrating the signal, circuit dividing the signal, denoising the signal, modulating the signal, filtering the signal, comparing the signal, compressing the signal, reconstructing the signal, converting the signal to data, extracting the data and processing the data. The data is then sent to a data control unit to be processed and then the data is displayed and communicated. FIG. 8 provides substantial detail about the preferred process of the present invention.

As illustrated in FIG. 8, raw data is collected from a user via a plurality of sensors in the enhanced data signal acquisition step. Raw data from the EEG sensor goes into the raw EEG input step and raw data from the ECG sensor goes into the raw ECG input step. An electronic amplifier then begins filtering usable data from unusable data. The usable data is then calibrated against known data to establish a common fingerprint for continued filtration and amplification. The circuit dividing step divides the data into separate megaHZ and more unusable data is filtered out. The preprocessing denoising then processes the raw filtered usable data into different frequencies. The EEG and ECG have standard fields of megaHZ. The amplitude modulator filters raw signals to identify higher megaHz. The amplitude modulator is part of and continues the filter, amplify, calibration process. The width modulator is also part of the filtering, amplifying and calibrating process. The width modulator removes unwanted noise from usable data in the development of the user's profile. The position modulator begins the process of developing an individual user's profile from usable data that has been filtered a plurality of times to remove unusable data. The inverse multiplexer (IMUX) uses an algorithm to aggregate data channels together. The database matched filtering builds a new database from the user's profiles. The comparative unit compares the profiles. The compression unit compresses the data into a usable signal. After the compression, there is a reconstructed usable readable signal. This is preferably displayed as a graph. The digital converter converts an analog signal into a digital signal that the computer can work with. The computer and/or software program then analyzes the collected data. The color processor separates the data of differing megaHz into differing colors. These colors light up an LED display in the domed brain shaped cover of the apparatus so that a user administering the test can see the different areas of the brain that are generating the frequency signal.

The external power is, for example, plugging the computer into an electrical socket. The internal power refers to the battery of the computer and an additional battery included in carrying case 20.

In the data control unit, the data output is displayed on a monitor capable of printing measurements. The microprocessor is an internal memory for comparison of data and saves information on the computer. The internal memory saves the data and the comparisons in the computer. The program memory holds the software mathematical algorithms (RAM). These algorithms are necessary for the display of the data as output on the monitor. The data memory is stored in the hard drive of the computer (ROM). The data member connects to the server/network, printer, storage unit and display-audio output.

The communication devices are input devices for communication with the information gathered by the apparatus.

Embodiments of the present invention comprise expanded neurofeedback stimulation and treatment apparatuses that allow performance of high frequency electroencephalogram (EEG) neural feedback to effect stimulation of higher frequency domains across multiple brain regions. The embodiments integrate a cardiac electromagnetic signal with a electroencephalographic signal through a mathematical algorithm that links the base cardiac signal to increasingly higher frequency brain electromagnetic signals, allowing conventional feedback in the 0-30 Hz domain (but expanded over multiple brain regions) as well as higher frequency feedback in the frequency domains from about 30 to about 1000 Hz with an optional embodiment to even higher frequency realms (e.g., 1000 Hz and above). Enhanced feedback through the cardiac-brain coupling is also achieved.

One embodiment of the present invention uses an apparatus for measurement and then incorporates software-based neural feedback aspects across full frequency zones and multiple brain regions along with feedback based on mathematical coupling of cardiac-brain electrical signals through software-based mathematical algorithms. This embodiment expands the measurement capability to the realm of medical treatment based on new technology for neural-cardiac feedback. The apparatus of this embodiment is preferably convenient and light-weight and is more preferably a peripheral USB apparatus with wireless technology. The apparatus allows a greater range of neural feedback options (higher frequency, expanded brain regions) along with cardial-neural feedback and thus expands use or treatment capabilities of existing technology and allows improved use or treatment for numerous injuries, illnesses and treatments, including but not limited to traumatic brain injury, psychiatric illness and cardiac disease. This embodiment of the present invention is used as a recovery apparatus by providing improved use or treatment for the disorders listed above for which conventional neural feedback has already proven successful. An embodiment of the present invention is used as a prevention apparatus by strengthening the functional state of a healthy brain and heart to impart resistance to trauma and disease.

The expanded neural-cardial feedback technology of the embodiments of the present invention allow correction of electromagnetic transcription errors in brain and cardiac function induced by trauma or various neuropsychiatric and cardiac disease states. Neuro-cardial feedback technology effects change in brain and cardiac function through re-connection of the cardiac and neural electromagnetic field states. A user learns to adjust the electric field states via feedback while watching a computer-generated image and changing it through mental concentration to the corrected state. An example of a use of the apparatus of the present invention is that it reverses the electromagnetic transcription errors in the brain and heart caused by trauma or disease. The term “transcription error” describes an abnormal field state (mathematical summation of the cardiac and brain electric fields) induced by trauma or disease. Through feedback loops, the patient readjusts the cardial-neural electromagnetic fields to a corrected balance point. The root of these transcription errors lies in the disconnection between the cardiac and neural signals that has not been addressed by previous technology and treatment methods. Neuro-cardial feedback technology effects change through re-connection of the cardiac and neural electromagnetic field states. An example of a use of the apparatus of the present invention is that it reverses the electromagnetic transcription errors in the brain and heart caused by trauma or disease. The cardial-neural electromagnetic fields are thus returned to a corrected balance point.

### Measuring Apparatus Example

The following is a non-limiting example of a measure apparatus.

Referring to FIGS. 7A-7H, the apparatus is preferably a flip form factor and is preferably constructed of a durable material. An example of a durable material is aluminum, preferably elemental silver aluminum with two flip up bays stacked one above the other. The bays open on zinc alloy hinges life tested to 20,000 times and torsion decay less than about 15%. The upper compartment cap has an integrated dome, preferably a three dimensional Gorilla Glass 2 (brain shaped) dome, that is used as the storage cover for the 65 W AC adapter, a reference point sensor and EEG/ECG sensors. The 3D brain dome preferably includes a LED lighting array designed to pulse with EEG/ECG data adding limited signal observation possibilities from a short distance. The lower compartment is used as the laptop cradle. A bay divider panel serves as the lower bay laptop cradle cover as well as a cradle for the 65 W AC adapter and EEG/ECG sensors. The top side of the divider panel supports high strength plastic form fitting and compact spring cradles designed to hold the AC adapter and EEG/ECG sensors in place while making them easy to insert and remove from the cradle.

In this example, the bay divider panel also houses a 4.0 MP HD dual webcam mounted on the opening end. The laptop cradle holds the computer securely in place with the One Touch™, computer lock down system that utilizes a 4″ vacuum mount that attaches to the back of the laptop.

The apparatus of one embodiment of the present invention comprises a docking pod that is designed to keep all components safe, secure, and ready to use in numerous situations. In a non-limiting example, a laptop bay provides the following computer interface options to communicate with the laptop: 1× audio video—HDMI-19 pin HDMI Type A, 1×VGA, 1×Hi-Speed USB-4 pin USB Type A, 1× audio-headphones/microphone-mini-phone 3.5 mm, 1× network-Ethernet 10Base-T/100Base-TX RJ-45, 2× Hi-Speed USB-4 pin USB Type A, 1× docking port replicator.

The docking pod expands the capabilities of the laptop with EEG/ECG analysis software installed on a built-in 128 GB solid state hard drive, a 1 GB graphics card, stereo speakers, 2.1 stereo sound, and a built-in microphone. The laptop monitor is used for visual display of the EEG and EKG tracings which are preferably in the form of voltage vs. time.

An additional 56-watt-hour 6-cell lithium ion backup battery with the following additional external interface devices add functionality to the laptop: 1×8× speed (DVD [symbol] ±R DL/DVD [symbol] ±RW/CD-RW), 1×802.11b/g/n, 1× Bluetooth 3.0 HS, 1×RJ-45 Ethernet 10Base-T/100Base, 2× Thunderbolt ports, 4×USB 2.0 ports, 1×SDXC card slot and a high-performance 1500 rpm fan cooling system to prevent laptop overheating. A retractable carrying handle is optionally built-in to the underside of the pod to ensure relocation is quick and easy while the lock slot optionally ensures it is secure.

User interface with the EEG/ECG analysis software is preferably via wired or wireless sensor EEG headset and EKG bioelectrode sensors. The headset can be, for example, a Bluetooth® high resolution neuro-signal acquisition wireless sensor headset and a EKG insulated bioelectrode sensors. The EEG headset has about 14 saline sensors that offer optimal positioning for accurate spatial resolution and gives the user total range of motion. The ECG is the smallest and lightest on the market. It is attached over right atrium of the subject's chest and transmits real-time ECG wireless signals to the laptop or over the Internet to a computer server that has been programmed to identify abnormal patterns.

One embodiment of the present invention comprises software that optionally comes pre-installed on an internal copyright protected 128 GB solid state hard drive. The housing construction of this embodiment is optionally an elemental silver aluminum with a 3D Gorilla Glass 2 (brain shaped) dome for probe set and power storage, a 45W power adapter, A/C wall plug and power cord, synchronized led brain wave lighting integrated into the brain dome, 4.0 MP HD dual webcams, 1 GB graphics card, 6-cell lithium ion battery, 8× (DVD±R DUDVD±RW/CD-RW), One Touch™ computer lock down system, stereo speakers, 2.1 stereo sound, built-in microphone, 802.11n Wi-Fi, matched filtering technology, Bluetooth® wireless technology, a high resolution neuro-signal acquisition wireless 14 sensor headset, ECG insulated bioelectrode sensor and reference point probe that attaches to the ear.

A long-recognized limitation of existing bio/neuro-feedback techniques is maintenance of the corrected state of balance achieved during treatment, outside the need for ongoing treatment sessions. An embodiment of the present invention comprises one or more portable reset apparatuses to remove this limitation. The reset apparatuses are an extension to the expanded neurofeedback stimulation and treatment apparatus. The portable reset apparatuses are carried or worn by an individual and activated by the individual at will through manually pressing a small switch or button on a surface of the reset apparatus.

### Reset Apparatus

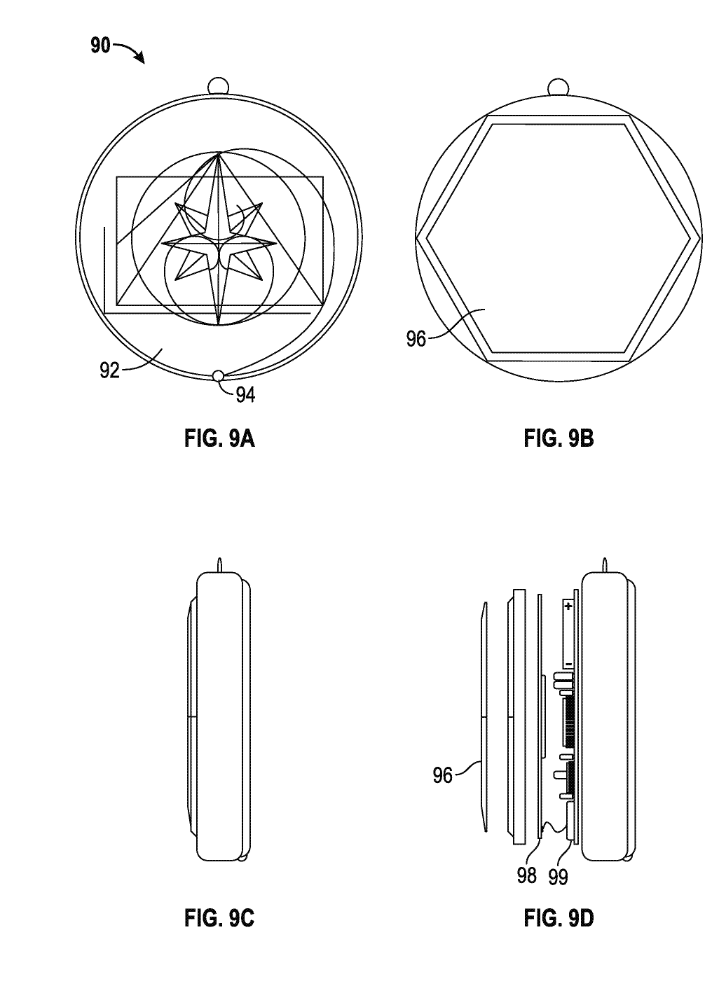

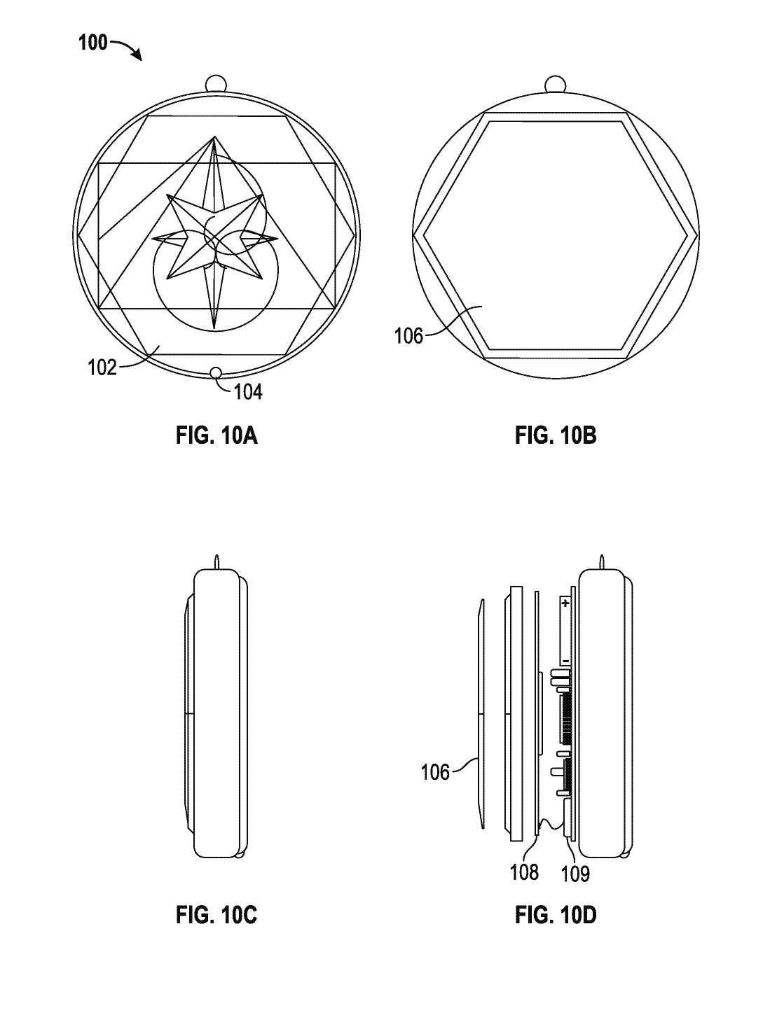

Referring to FIGS. 9A-9D and 10A-10D, an embodiment of the present invention comprises prevention reset apparatus 90 for prevention which acts as a reminder of the balanced cardial-neural field state in normal individuals. Recovery reset apparatus 100 is for recovery and/or treatment, which acts as a stabilizer to rebalance the cardial-neural field state in individuals recovering from trauma or disease. Reset apparatuses 90 and 100 comprise micro-circuitry that delivers a tactile vibratory pattern to a hand of an individual. A vibratory pattern is set by specific mathematical algorithms designed to provide a tactile tonal key to the parietal lobe of the brain which works with the electromagnetic field in delivering a reset pattern. The mathematical algorithms for prevention and recovery reset apparatuses 90 and 100 are different and designed to result in functional coding differences for either prevention (remembrance of the balanced pattern) or recovery (restabilization of the pattern to balance). The vibratory patterns act beyond the tactile “feel” of the case to cement the tactile coding in the parietal lobe.

Reset apparatuses 90 and 100 of embodiments of the present invention involve technology which activates in three steps. This process acts as a map back to a balanced cardial-neural state. A prevention reset apparatus acts as a memento of the balance cardial-neural field state in a typical individual. A recovery reset apparatus acts as a stabilizer to rebalance the cardial-neural field state in individuals recovering from trauma, illness or disease.

### Step 1: Tactile Stimulus

In one embodiment of the present invention, case 92 for reset apparatus 90 is constructed of a chemical composition, which when charged via a manually activated microcircuitry, yields a specific electromagnetic field pattern around an individual that amplifies the cardial-neural field linkage. The chemical composition includes, but is not limited to a silver-germanium alloy (Argentium silver), silver, copper and/or trace amounts of germanium (or the like) coupled with a piezoelectric quartz crystal charged with nanoparticles of the platinum group elements and palladium group elements, including but not limited to, rhodium and iridium. Prevention reset apparatus 90 (“Reminder”) comprises a simpler, cleaner surface design for case 92. Recovery reset apparatus 100 (“Stabilizer”) comprises a more complex surface design for case 102. The different example surface designs present distinct tactile stimuli to the parietal lobe of the brain that result in functional coding differences for either prevention (remembrance of the balanced pattern) or recovery (restabilization of the pattern to balance).

Prevention reset apparatus 90 comprises reset button 94, disc 96, frequency disc 98 and circuit board 99. Disc 96 is a cover, preferably a metal cover and more preferably a cover made of silver. Frequency disc 98 produces a vibration that reinforces or resets a user that has been through a clinical program. Prevention reset apparatus 90 provides a treatment to get a user back on track if he/she feels the need. Reset button 94 activates circuit board 99 which sends a vibration to frequency disc 98, while the user is holding disc 96 against his/her body. The vibration and pressure of holding disc 96 against the body sends a reset message through the body and to the brain. Circuit board 99 stores a vibration as a sound wave and generates an electrical current that pushes frequency disc 98 back and forth. The vibration is transmitted to disc 96 which is pressed against user's chest, creating an internal vibratory pattern.

Recovery reset apparatus 100 comprises reset button 104, disc 106, frequency disc 108 and circuit board 109. Recovery reset apparatus 100 functions the same a prevention reset apparatus 90. Disc 106 is a cover, preferably a metal cover and more preferably a silver cover. Frequency disc 108 produces a vibration that reinforces or resets a user that has been through a clinical program. Recover reset apparatus 100 is preferably a treatment device to get the user back on track if they felt the need. Reset button 104 activates circuit board 109 which sends a vibration to frequency disc 108, while the user is holding disc 106 against his/her body. The vibration and pressure of holding disc 106 against the body sends a reset message through the body and to the brain. Circuit board 109 stores a vibration as a sound wave and generates an electrical current that pushes frequency disc 108 back and forth. The vibration is transmitted to disc 106 which is pressed against the user's chest, creating an internal vibratory pattern.

### Step 2: Vibrator Stimulus

Reset apparatuses 90 and 100 have micro-circuitry that delivers a tactile vibratory pattern to the hand of an individual. A vibratory pattern is set by specific mathematical algorithms designed to provide a tactile tonal key to the parietal lobe of the brain which works with the electromagnetic field in delivering a reset pattern. The mathematical algorithms for the prevention and recovery reset apparatuses are different and designed to result in functional coding differences for either prevention (remembrance of the balanced pattern) or recovery (restabilization of the pattern to balance). The vibratory patterns act beyond the tactile “feel” of the case to cement the tactile coding in the parietal lobe. Vibratory patterns are sensed via skin receptors called Pacinian corpuslces that are separate from the light touch sensors activated by the static “feel” of the case. The receptive field within the somatosensory cortex of the Parietal lobe for vibration sense is distinct anatomically from that for light touch. The vibratory pattern serves to deliver stimulation to a broader area of the Parietal lobe opening up pathways that are important to a third step in the process. Steps one and two thus serve as a Parietal lobe static electric field alteration and a vibratory tonal key.

### Step 3: Supra-Auditory Signal

A high-frequency auditory signal is delivered by the micro-circuitry of the reset apparatus, which although above the normal detection limit of the human ear, nevertheless delivers a supra-auditory vibratory pattern to the temporal lobes of the brain thus acting as a temporal tonal key which works in conjunction with the electric field and parietal tonal key to deliver a fully reset cardial-neural pattern. Once again, the frequency pattern is different for prevention and recovery reset apparatuses acting as either a reminder or stabilizer of the balanced cardial-neural field pattern.

The three stimuli of the electromagnetic field (tactile stimulus, vibratory stimulus, and supra-auditory signal) when deployed in conjunction act to reset an individual back to a balanced state of a connected cardial-neural field. The reset apparatuses of the present invention are, as a non-limiting example, approximately 1-5 cm and preferably about 3 cm in diameter, and about 0.1-1.0 cm thick and preferably about 0.5 cm thick.

### Sound and Light-based Transformer Unit

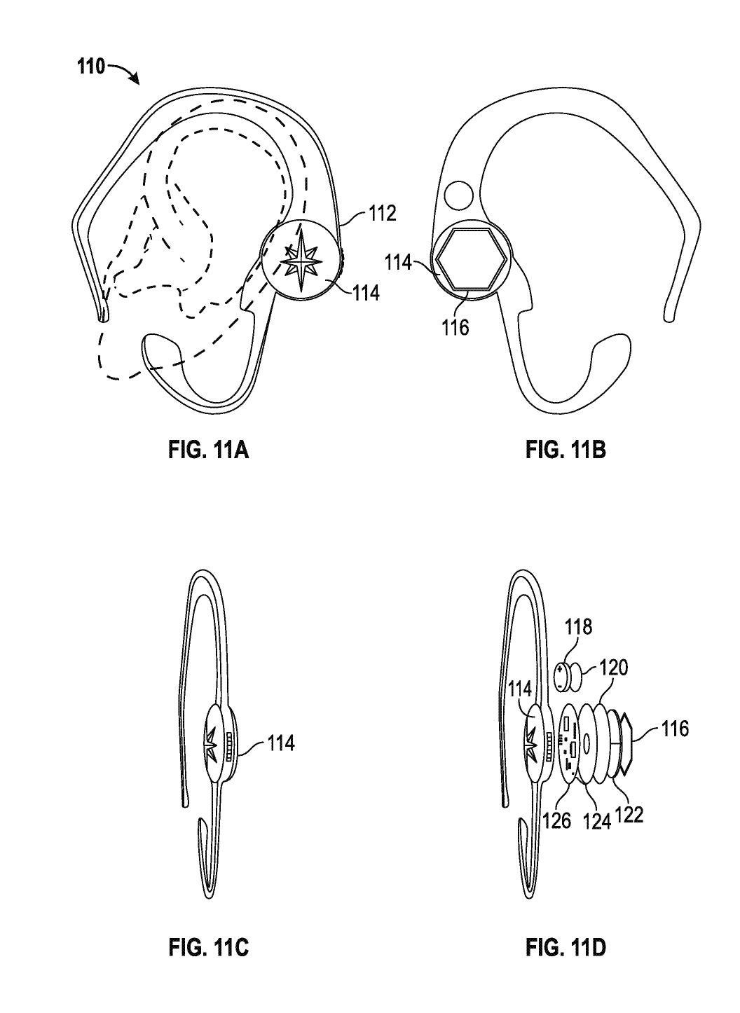

Referring to FIGS. 11A-11D, another embodiment of the present invention comprises sound- and light-based transformer unit 110 that carries a functional state of a cardial-neural field beyond the level of neural based chemical electrodynamics. Transformer unit 110 preferably fits to a user's ear, for example, similar to an earpiece. Transformer unit 110 activates both the cardiac and neural glial cells into a functional syntitium that allows thought to progress from the limitations of electromagnetic field fluctuations to quantum photon-based light processes. Field potentials which form the current limited state of brain function are transformed to light potentials. The brain is then allowed to progress at increasing functional acceleration by using light-based principles beyond simple electromagnetic/electrochemical signaling.

Sound- and light-based transformer unit 110 comprises earpiece 112, multi-controller 114. Multi-controller 114 preferably comprises piezoelectric disc 116, preferably a piezoelectric quartz crystal disc, one or more batteries 118, one or more covers 120, light disc 122, frequency disc 124 and circuit board 126. Transformer unit 110 comprises circuit board 126 that is preferably battery powered. Circuit board 126 produces light and sound from embedded firmware. The firmware interfaces with frequency disc 124 for sound and light-emitting diode disc 122 to produce light. The light and sound pass thru piezo quartz crystal disc 116 which adds to the over-all effect when ear piece 112 is pressed against the mastoid bone behind the ear. Multi-controller 114 controls the on/off function of circuit board 126 by pressing in to turn it on and pressing it again to turn it off. It also rotates to increase or lessen the sound vibrations and light pulses.

Transformer unit 110 of an embodiment of the present invention provides specific mathematical algorithms encoded in the sound of music (musical algorithms) which act as a catalytic tonal key. Transformer unit 110 also comprises a photonic activator unit, preferably a seed crystal, that acts as a direct light-based thought resonance generator through direct photonic field activation of the pineal gland as the typical retinal visual pathways are bypassed. A non-limiting example of a photonic activator unit comprises a green obsidian based crystalline unit charged with sound-generated nanoparticles of the platinum group elements, including but not limited to, rhodium and iridium. The sound unit preferably activates the photonic function of the crystalline matrix.

The field interaction of the musical algorithms from the sound unit coupled with the photonic activation of the crystalline unit carry thought processes from the bound realm of chemical electrodynamics to the limitless realm of unbound thought through the activation of the sound and light-based transformer unit functioning of the cardial-neural glial cell syncytium. In one embodiment of the present invention, the sound unit fits the left ear, see, for example FIG. 11A-11D.

### Thought Amplification Apparatus

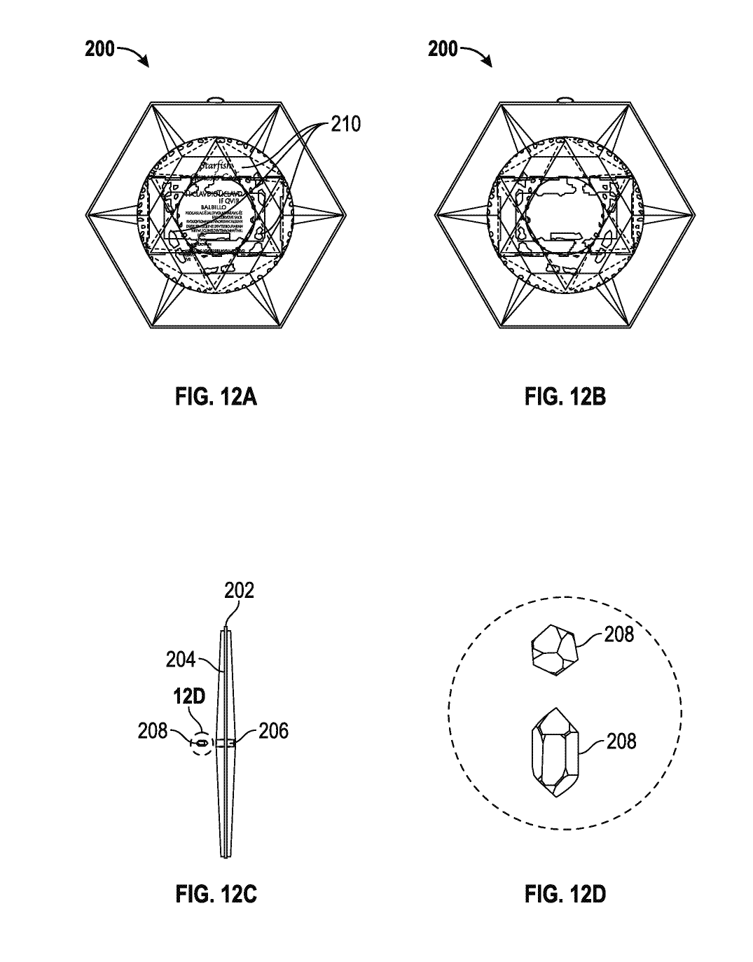

Referring to FIGS. 12A-12D, an embodiment of the present invention comprises thought amplification apparatus 200 and method of use of thought amplification unit 200. This embodiment preferably transforms consciousness by further elevation of sound- and light-based transformer unit 110 functioning of the cardial-neural-glial cell syncytium affected via sound and light-based transformer unit 110. Thought amplification apparatus 200 and method embodies a complete thought-based holographic generator of manifestation, contains all steps within its matrix.

The apparatus employs the technology of thought amplification by stimulated resonance, preferably with a quartz crystal having elements from the platinum group thought amplification by stimulated resonance (Q-TASR). In an analogy to existing technology, the thought amplification apparatus and method acts as a “thought laser” to provide resonant amplification of the force of thought.

Thought amplification apparatus 200 preferably comprises loop 202, band 204, laser tunnel 206 and seed crystal 208. Seed crystal 208 is preferably a quartz-platinum group crystalline structure.

### Thought Amplification Apparatus Description Example

Thought amplification apparatus 200 is preferably a hexagon or other shape having a crystal, preferably, for example, a hand-held green obsidian hexagon (for example, dimensions being length of about 6 cm, sides of the hexagon about 3 cm, depth about 2 cm) with seed crystal 208, preferably quartz, (ovoid, for example, of about 0.4 cm×0.2 cm×0.1 cm) embedded within its center. The obsidian hexagon is preferably molded as a single holistic unit. Laser tunnel 206 is formed via laser tunneling which is preferably used to insert seed crystal 208 into the center and reseal the obsidian; thus maintaining structural integrity. The edges of the obsidian hexagon are encircled by a band, preferably a narrow Arengtum silver band and sound-charged with platinum group and rare earth (lanthanide) elements. Band 204 comprises circular loop 202 for hanging on a chain. Seed crystal 208 comprises sound-generated platinum group elemental nanorods and aquo-acoustical algorithms embedded through the sound of water in its core.

Surface etchings 210 on the hexagon impart specific mathematical algorithms through its design termed surface coding. Surface etchings 210 act as visual and tactile catalysts. Etchings 210 embedded into the surface (see FIGS. 12A-12D) show the basic ancient geometric structures reflected in the earth from the stars. Built into this etching 210 is identifiable and familiar language of symbolism. These geometric patterns toggle the human mind to remember its most innate indigenous awareness, the way of defining and using the DNA at will. Embedded in etchings 210 of geometric shapes are mathematical algorithms that comprise the elemental force of certain platinum group elements and certain lanthanide elements.

Band 204, preferably an argentum silver band, acts as a surface geometric amplifier through thought amplification apparatus 200 and the embedded Pt-group and/or lanthanide group elemental energies. This geometric order and the use of certain platinum group and lanthanide group elements in their high spin state creates thought amplification by stimulated resonance state.

Thought amplification apparatus 200 is preferably activated via light, heat and the human cardial-neural electromagnetic field when held in the palm. Interaction between thought amplification apparatus 200 and the pineal gland-temporal lobe-cardiac arc creates the thought amplification by stimulated resonance state, for example, the quartz-based thought amplification via stimulated resonance state.

Seed crystal 208, preferably a quartz seed crystal is charged with sound-generated nanorods of the platinum group (Pt group) and/or palladium group elements, such as, for example, rhodium and iridium which impart a liquid crystalline structure with specific geometry to the center of seed crystal 208. In addition, aquo-acoustical algorithms are coded through the sound of water and are embedded within seed crystal 208 core.

In one example, the ordered geometric pattern of the seed crystalline matrix interacts with the entropic structure of the amorphous obsidian, band 204 and geometric surface etchings 210 activating the mathematical algorithms contained within surface etchings 210 and the aquo-acoustical algorithms coded through thought amplification apparatus 200 and sound of water in crystal 208 core to generate a specific tonal key that links thought amplification apparatus 200 to the pineal-temporal-cardiac field arc pre-activated via application of sound and light based transformer unit 110.

The platinum group nanorods impart specific geometric form to the liquid crystalline center that catalyzes a specialized super-conductor gravimetric state of atomic nuclear gravi-magnetic resonance within the crystalline matrix. The interaction between this resonant state and the mathematical algorithms of surface etchings 210 and band 204 allow reflection of the force of thought. Feedback activation of specific vibrational tones within the pineal gland-temporal lobe arc of the brain and the heart glial light fields results in stimulated resonance between the force of thought and the quartz matrix allowing collapse of time; hence making complete geometric manipulation of space and form accessible. It is the linkage of light-sound-geometry-obsidian-(for example, argentum silver-Pt/Lanthanide group elemental) entropic high spin state to the activated pineal-temporal arc and heart fields that creates the state of stimulated resonance.

As a non-limiting example, thought amplification apparatus 200 integrates three basic components:
(1) quartz with elements from the platinum group crystalline structural geometry using an internal quartz seed crystal charged with Pt-group elements (Q-TASR unit);
(2) entropic high spin state activated through the external structure of the thought amplification apparatus using linkages of obsidian-[argentum silver-Pt group-lanthanide group elements] and geometric surface etchings 210 (surface unit);
(3) vibratory tone key created through sound elements and mathematical algorithms held within seed crystal 208, surface etchings 210 and external silver band 204 (tonal unit).

The components listed above, when activated via light, heat and the human cardial-neural electromagnetic field when held in the palm of a hand provide a link to the pineal-temporal-cardiac field arc through the catalysis of the light-activated visual pathways of the retinal-pineal-neural-cardial connection.

Seed crystal 208 contains specifically embedded mathematical algorithms within its structure. Seed crystal 208, as with other crystalline substances, contains inherent mathematics within its ordered structure. Seed crystal 208 is preferably placed in water and specific acoustical patterns are pulsed through the water which then subtly alter the inherent mathematical structure of the crystal into a format that contains, not only the acoustical pattern of water, but also the mathematical patterns within the acoustical pulses sent through the water (i.e., aquo-acoustical algorithms). Seed crystal 208 is thus enhanced by embedding the patterns of the acoustical pulses and water within its matrix and, which when activated and linked to two other components, nanorods of platinum group elements embedded with seed crystal 208 and musical patterns pulsed through earpiece 112 of sound and light based transformer unit 110, serves as a catalytic key activating human DNA in which the appropriate musical tone is embedded. Seed crystal 208 comprises one or more nano-rods of platinum group elements (not shown). The presence of the nanorods within the crystalline matrix of seed crystal 208 alter the mathematical pattern inherent in the crystal structure to allow its subsequent modification via the pulsed acoustic patterns instilled with seed crystal 208 immersed in water. Once this has been accomplished, seed crystal 208 is assembled in earpiece 112 which also comprises specific musical patterns that play into a user's ear thus coded in the acoustical reception areas of the temporal lobe. The resulting enhanced sound and light-based transformer functioning of the glial syncytium catalyzes a second star burst pattern, an octave higher, carrying the brain across the photonic bridges from light-based function to supra-light function. Thought becomes unbound and full access to the library of knowledge is achieved. Thought amplification apparatus 200 and method preferably allows progression of thought from field potential to light potential to unbound limit-less potential. Thus, a user achieves their natural state of being, unbound thought without limits.

Embodiments of the present invention carry advanced feedback processes linking light, sound and synchronization with the individual heart beat through a thought resonant process. Thought amplification apparatus 200 is a resonant acoustical photonic apparatus.

The method of an embodiment of the present invention comprises a step-wise progression involving measurement of higher frequency electromagnetic patterns in the brain coupled with synchronous mapping of the cardiac electromagnetic field. A next step employs neural-cardial feedback to activate higher frequency electromagnetic functioning in the brain and to link brain/heart electromagnetic fields into a balanced state. Reset apparatuses 90 and 100 preferably allow continuous reminding/restabilization of the balanced field patterns. Sound and light transformer unit 110 then takes the function of the cardiac/brain fields beyond electromagnetic to light-based (photonic) function through activation of the glial cell syncytium. Thought amplification apparatus 200 links technology incorporating thought amplification by stimulated resonance, entropic spin state geometries and tonal (sound-based) elements.

Note that there is also an optional proprietary educational program for application of sound and light-based transformer unit 110 and thought amplification apparatus 200. The educational program is preferably loaded onto a hand-held computer, such as, for example, an i-Pad and coupled to music and visual displays.

Although the invention has been described in detail with particular reference to these preferred embodiments, other embodiments can achieve the same results. Variations and modifications of the present invention will be obvious to those skilled in the art and it is intended to cover in the appended claims all such modifications and equivalents. The entire disclosures of all references, applications, patents, and publications cited above are hereby incorporated by reference.

What claimed is:

1. An apparatus for measuring electromagnetic activity in a user's brain and heart comprising:
a computer;
a display screen;
a software program for rapid measurement and digital display of the user's electromagnetic brain and heart frequencies above about 500 Hz;
a plurality of EEG sensors;
an EKG sensor; and
said plurality of EEG sensors and said EKG sensor connected to said computer.

2. The apparatus of claim 1 further comprising an integrated carrying case for storing said EEG sensors and said EKG sensor.

3. The apparatus of claim 1 further comprising an all-in one carrying case for said computer and said sensors.

4. The apparatus of claim 1 further comprising a headset having said plurality of EEG sensors disposed on said headset.

5. The apparatus of claim 1 wherein the user's electromagnetic brain and heart frequencies are measured and displayed between about 500 and about 1000 Hz.

6. The apparatus of claim 1 wherein said apparatus is portable.

7. The apparatus of claim 1 wherein said computer comprises a laptop.

8. The apparatus of claim 1 wherein said computer comprises a tablet.

9. The apparatus of claim 1 wherein said plurality of EEG sensors and said EKG sensor are connected to said computer via a peripheral USB device.

10. The apparatus of claim 1 wherein said plurality of EEG sensors and said EKG sensor are connected to said computer wirelessly.

11. The apparatus of claim 1 wherein said software program continuously measures the user's electromagnetic brain and heart frequencies for biofeedback.

12. A method for measuring electromagnetic activity in a user's brain and heart comprising:
placing a plurality of EEG sensors on a user's head;
placing an EKG sensor on the user's chest;
transmitting a plurality of EEG signals to a computer server;
transmitting an EKG signal to the computer server;
measuring the user's electromagnetic brain and heart frequencies above about 500 Hz; and
displaying the user's electromagnetic brain and heart frequencies on a display screen.

13. The method of claim 12 wherein the measuring of the user's electromagnetic brain and heart frequencies above about 500 Hz is performed in real-time.

14. The method of claim 12 further comprising integrating the heart electromagnetic signal with the brain electromagnetic signal.

15. The method of claim 12 further comprising detecting an abnormal pattern in either the heart electromagnetic signal or the brain electromagnetic signal.

16. The method of claim 12 wherein the EEG signals and the EKG signal is transmitted to a computer.

17. The method of claim 12 wherein the EEG signals and the EKG signal is transmitted over the Internet to the computer server.

18. The method of claim 12 further comprising tracking the user's electromagnetic brain and heart frequencies above about 500 Hz.

19. The method of claim 12 wherein the user's electromagnetic brain and heart frequencies are measured between about 500 and about 1000 Hz.

20. The method of claim 12 comprising continuously measuring the user's electromagnetic brain and heart frequencies above about 500 Hz for providing biofeedback.
